(19) United States
(12) Patent Application Publication
(10) Pub. No.: US 2022/0288332 A1
(43) Pub. Date: Sep. 15, 2022

(54) OXYGEN AND NON-INVASIVE VENTILATION THERAPY MONITORING APPARATUS

(71) Applicant: MaRI Co., Ltd., Kyoto (JP)

(72) Inventors: Hirofumi TAKI, Kyoto (JP); Kazuo CHIN, Kyoto (JP)

(73) Assignee: MaRI Co., Ltd., Kyoto (JP)

(21) Appl. No.: 17/639,763

(22) PCT Filed: Sep. 2, 2020
(86) PCT No.: PCT/IB2020/000705

§ 371 Date: Mar. 2, 2022

Related U.S. Application Data
(60) Provisional application No. 62/894,953, filed on Sep. 2, 2019.

(51) Int. Cl. A61M 16/00; A61M 16/10

(52) U.S. Cl. CPC A61M 16/024; A61M 16/0051; A61M 16/1005; A61M 2016/0039; A61M 2205/3375; A61M 2230/42; A61M 2205/3553; A61M 2230/46; A61M 2205/15; A61M 2205/44; A61M 2205/582; A61M 2205/587; A61M 2205/581

(57) ABSTRACT

A flow monitoring apparatus includes one or plural hydrophones that receive plural sounds transmitted through a tube wall and/or inside a tube, and a controller including circuitry which converts the plural sounds received by one or plural hydrophones to received signals, extracts flow information from the received signals, evaluates flow rate in a tube, and transmits the volume flowing in a tube per unit of time.

### CROSS-REFERENCE TO RELATED APPLICATIONS

The present application is based upon and claims the benefits of priority to U.S. Provisional Application No. 62/894,953, filed Sep. 2, 2019. The entire contents of the above applications are incorporated herein by reference.

### TECHNICAL FIELD

The present invention is directed to a monitoring apparatus that monitors oxygen and non-invasive ventilation therapy.

### BACKGROUND ART

Oxygen therapy and non-invasive ventilation therapy may be used for the prevention of hypoxia in the intensive care unit and the perioperative care. Pulse oximetry is a monitoring apparatus to detect the occurrence of hypoxia (NPL 1). Multiple wavelength pulse oximetry can improve the performance of pulse oximetry (NPL 2). However, a pulse oximetry detects hypoxia with some delay from the oxygen or air leak, because oxygen leak for a certain time causes hypoxia. Flow sensors and/or pressure sensors are employed to monitor the delivery of oxygen to a patient (PL 1, PL 2, PL 3). Although there are several methodologies that estimate the magnitude of oxygen leak using flow sensors and/or pressure sensors, the detection of oxygen leak is still a difficult problem.

### Citation List Patent Literature

PL 1 S. Schatzl, D. C. C. Martin, “Monitor for CPAP/ventilator apparatus,” US20090020120A1.
PL 2 M. A. A. Rashad, P. W. Belanger, B. R. Bielec, “Nasal pressure sensor oxygen therapy device,” U.S. Pat. No. 7,013,898B2.
PL 3 J. W. Beard, “Oxygen facemask with capnography monitoring ports,” US20130060157A1.

### Citation List Non Patent Literature

NPL 1 A. Jubran, “Pulse oximetry,” Crit Care, 2015; 19: 272.
NPL 2 T. W. L. Scheeren, F. J. Belda, and A. Perel, “The oxygen reserve index (ORI): a new tool to monitor oxygen therapy,” J Clin Monit Comput, 2018; 32:379-389.

### SUMMARY OF THE INVENTION

According to an aspect of the present invention, a flow monitoring apparatus includes one or plural hydrophones that receives plural sounds transmitted through a tube wall and/or inside a tube, and a controller including circuitry which converts the plural sounds received by one or plural hydrophones to received signals, extracts flow information from the received signals, evaluates flow rate in a tube, and transmits the volume flowing in a tube per unit of time.

According to another aspect of the present invention, an airflow monitoring apparatus includes at least one hydrophone that receives plural sounds transmitted through a tube wall and/or inside a tube, and a controller including circuitry which converts the plural sounds received by one or plural hydrophones to received signals, extract airflow information from the received signals, evaluate airflow rate in a tube, and transmit the information of air volume flowing in a tube per unit of time.

According to still another aspect of the present invention, a breathing monitoring apparatus includes at least one hydrophone that receives plural sounds transmitted through a tube wall and/or inside a tube, and a controller including circuitry which converts the plural sounds received by one or plural hydrophones to received signals, extract breathing information from the received signals, evaluate breathing pattern, respiratory rate, presence of breathing, and/or the airflow in a tube using the breathing information, monitor breathing pattern, respiratory rate, presence of breathing, and/or the airflow in a tube, and transmit the information related to respiration including breathing pattern, respiratory rate, and/or presence of breathing.

According to yet another aspect of the present invention, a breathing monitoring apparatus includes at least one hydrophone that receives plural sounds transmitted through a tube wall and/or inside a tube, and a controller including circuitry which converts the plural sounds received by one or plural hydrophones to received signals, extract breathing information from the received signals, store one or plural reference signals related to sounds transmitted through a tube wall and/or inside a tube, evaluate breathing pattern, respiratory rate, presence of breathing, and/or the airflow in a tube using the breathing information and the reference signals, monitor breathing pattern, respiratory rate, and/or presence of breathing using the airflow data evaluated from the breathing information, and transmits the information related to respiration including breathing pattern, respiratory rate, and/or presence of breathing.

According to yet another aspect of the present invention, an oxygen therapy monitoring apparatus includes at least one hydrophone that receives plural sounds transmitted through a tube wall and/or inside a tube, and a controller including circuitry which converts the plural sounds received by one or plural hydrophones to received signals, extract breathing information from the received signals, evaluate breathing pattern, respiratory rate, presence of breathing, and/or the airflow in a tube using the breathing information, monitor breathing pattern, respiratory rate, presence of breathing, and/or the airflow in a tube, and transmit the information related to respiration including breathing pattern, respiratory rate, presence of breathing, and/or airflow in a tube.

According to yet another aspect of the present invention, an oxygen therapy monitoring apparatus includes at least one hydrophone that receives plural sounds transmitted through a tube wall and/or inside a tube, and a controller including circuitry which converts the plural sounds received by one or plural hydrophones to received signals, extract breathing information from the received signals, store plural reference signals for different respiratory volumes and/or for air leakage conditions, evaluate breathing pattern, respiratory rate, presence of breathing, the airflow in a tube and/or air leakage using the breathing information and the reference signals, monitor breathing pattern, respiratory rate, presence of breathing, the airflow in a tube and/or air leakage, and transmit the information related to respiration including breathing pattern, respiratory rate, presence of breathing, the airflow in a tube and/or air leakage.

According to yet another aspect of the present invention, a non-invasive ventilation therapy monitoring apparatus includes at least one hydrophone that receives plural sounds transmitted through a tube wall and/or inside a tube, and a controller including circuitry which converts the plural sounds received by one or plural hydrophones to received signals, extract breathing information from the received signals, evaluate breathing pattern, respiratory rate, presence of breathing, and/or the airflow in a tube using the breathing information, monitor breathing pattern, respiratory rate, presence of breathing, and/or the airflow in a tube, and transmit the information related to respiration including breathing pattern, respiratory rate, presence of breathing, and/or airflow in a tube.

According to yet another aspect of the present invention, a non-invasive ventilation therapy monitoring apparatus includes at least one hydrophone that receives plural sounds transmitted through a tube wall and/or inside a tube, and a controller including circuitry which converts the plural sounds received by one or plural hydrophones to received signals, extract breathing information from the received signals, store plural reference signals for different respiratory volumes and/or for air leakage conditions, evaluate breathing pattern, respiratory rate, presence of breathing, the airflow in a tube and/or air leakage using the breathing information and the reference signals, monitor breathing pattern, respiratory rate, presence of breathing, the airflow in a tube and/or air leakage, and transmit the information related to respiration including breathing pattern, respiratory rate, presence of breathing, the airflow in a tube and/or air leakage.

According to yet another aspect of the present invention, a breathing monitoring apparatus includes at least one microphone that receives plural sounds transmitted through a tube wall and/or inside a tube, and a controller including circuitry which converts the plural sounds received by the at least one microphone to received signals, extract breathing information from the received signals, evaluate breathing pattern, respiratory rate, presence of breathing, and/or the airflow in a tube using the breathing information, monitor breathing pattern, respiratory rate, presence of breathing, and/or the airflow in a tube, and transmit the information related to respiration including breathing pattern, respiratory rate, and/or presence of breathing.

### BRIEF DESCRIPTION OF DRAWINGS

A more complete appreciation of the invention and many of the attendant advantages thereof will be readily obtained as the same becomes better understood by reference to the following detailed description when considered in connection with the accompanying drawings, wherein:

FIG. 1 is a schematic diagram of an airflow monitoring apparatus that employs a hydrophone with a reception circuit, an airflow information extraction block, an airflow-rate evaluation block, and an airflow information transmission block.

FIG. 2 is a schematic diagram of a signal processing device that extracts airflow information from received signals using at least one airflow information extraction filter, and evaluates information of air volume flowing in a tube per unit of time using at least one airflow rate evaluation filter.

FIG. 3 is a schematic diagram of a breathing monitoring apparatus that estimates breathing pattern, respiratory rate, presence of breathing, and/or the air volume flowing in a tube per unit of time.

FIG. 4 is a schematic diagram of a breathing monitoring apparatus using reference signals.

FIG. 5 is a schematic diagram of a breathing monitoring apparatus that estimates breathing pattern, respiratory rate, presence of breathing, and/or the air volume flowing in a tube per unit of time, and transmits alert information and/or emits warning sound when a breathing information evaluation block detects improper use or non-use of a breathing device.

FIG. 6 is a schematic diagram of a breathing monitoring apparatus that employs a microphone with a cover.

### DESCRIPTION OF EMBODIMENTS

The embodiments will now be described with reference to the accompanying drawings, wherein like reference numerals designate corresponding or identical elements throughout the various drawings.

A flow monitoring apparatus according to an embodiment of the present invention includes an apparatus that acquires a plurality of sounds transmitted through a tube wall 114 and/or inside a tube 100 in order to estimate the volume flowing in a tube per unit of time.

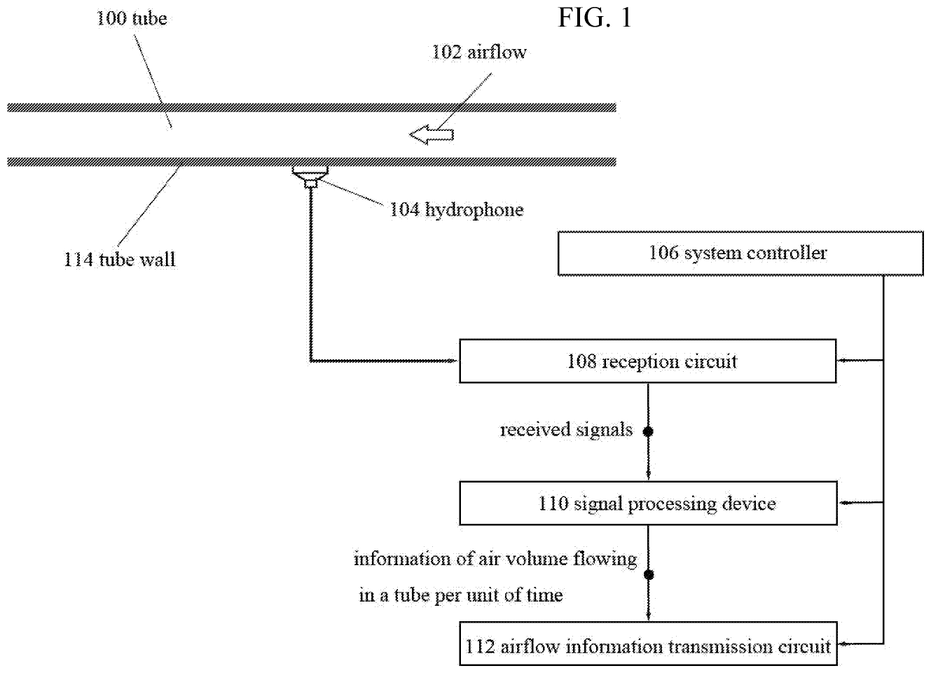

FIG. 1 shows a schematic diagram of an airflow monitoring apparatus that employs one or plural hydrophones 104 with one or plural reception circuits 108 that convert a plurality of sounds, created by several sound sources including friction between tube wall 114 and air, transmitted through a tube wall 114 and/or inside a tube 100 to a plurality of received signals, a signal processing device 110 that receives the received signals and evaluates airflow rate in a tube 100; and an airflow information transmission circuit 112 that transmits the information of air volume flowing in a tube per unit of time to an external device so that his/her attending physician and/or caregiver can monitor that information. A system controller 106 may be a computer that includes central processing unit (CPU) and a memory such as read-only memory (ROM) and random access memory (RAM). The CPU of the controller can be a single-core processor (which includes a single processing unit) or a multi-core processor. The computer may be a mobile device such as a personal digital assistant (PDA), laptop computer, field-programmable gate array, or cellular telephone. A signal processing device 110 may be a computer that includes central processing unit (CPU) and a memory such as read-only memory (ROM) and random access memory (RAM). The CPU of the controller can be a single-core processor (which includes a single processing unit) or a multi-core processor. The computer may be a mobile device such as a personal digital assistant (PDA), laptop computer, field-programmable gate array, or cellular telephone. A computer, that includes central processing unit (CPU) and a memory such as read-only memory (ROM) and random access memory (RAM), may include both the system controller 106 and the signal processing device 110. The system controller 106 may manage the passing and processing of the information.

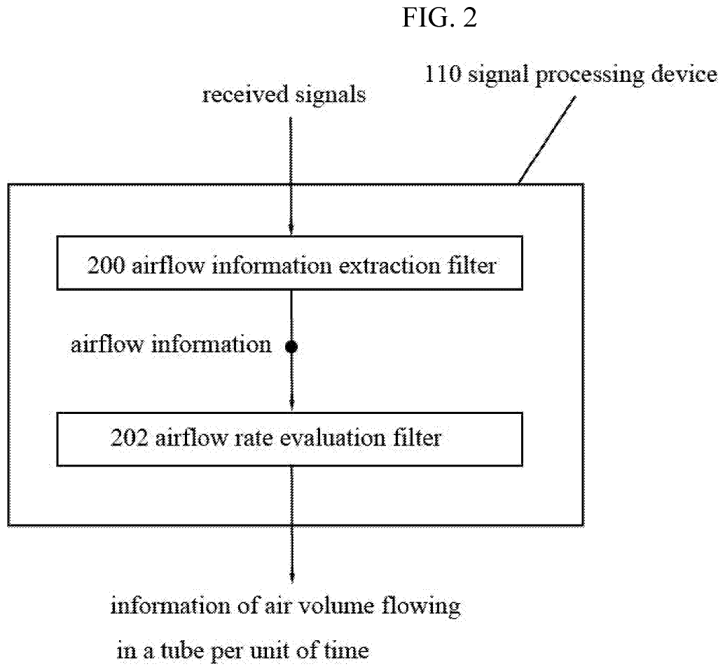

FIG. 2 shows a schematic diagram of the signal processing device 110 that extracts airflow information from received signals using at least one airflow information extraction filter 200, and evaluates information of air volume flowing in a tube per unit of time using at least one airflow rate evaluation filter 202. The airflow information extraction filter includes band path filter, high path filter, low path filter, and/or a filter that calculates RMS envelope of input signal. The airflow rate evaluation filter 202 includes the filter that converts the summation of signal intensity to the air volume, because the sound generated by airflow depends on the air volume flowing in the tube.

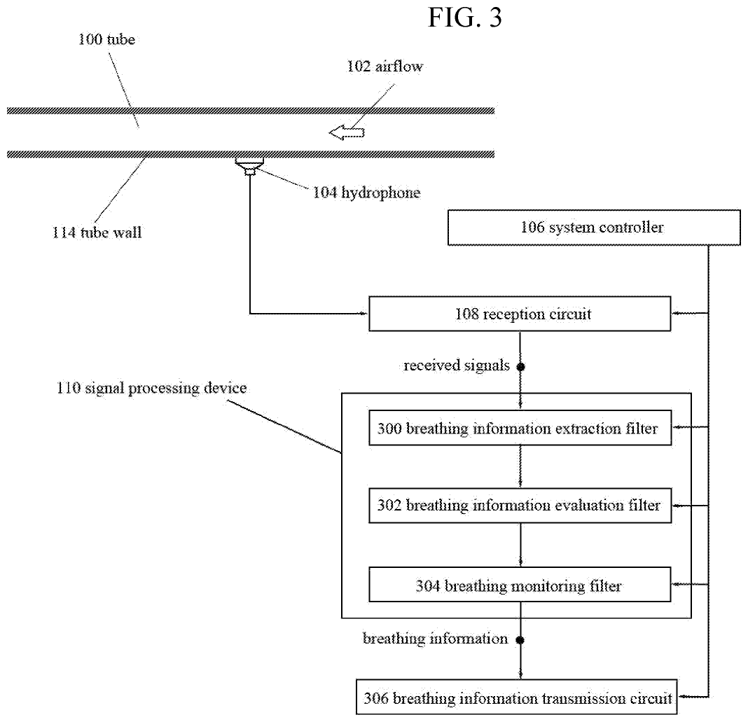

A flow monitoring apparatus according to an embodiment of the present invention can monitor breathing information in oxygen therapy and non-invasive ventilation therapy. FIG. 3 shows a schematic diagram of a breathing monitoring apparatus that estimates breathing pattern, respiratory rate, presence of breathing, and/or the air volume flowing in a tube per unit of time, comprising: one or plural hydrophones 104 with one or plural reception circuits 108 that convert a plurality of sounds transmitted through a tube wall 114 and/or inside a tube 100 to a plurality of received signals; a breathing information extraction filter 300 that extracts breathing information from a plurality of received signals acquired by one or plural hydrophones 104 with one or plural reception circuits 108; a breathing information evaluation filter 302 that evaluates breathing pattern, respiratory rate, presence of breathing, and/or the airflow in a tube using the breathing information extracted by the breathing information extraction filter 300; a breathing monitoring filter 304 that monitors breathing pattern, respiratory rate, presence of breathing, and/or the airflow in a tube; and a breathing information transmission circuit 306 that transmits the information related to respiration including breathing pattern, respiratory rate, and/or presence of breathing. For example, the tube 100 may be connected from a ventilator to a mask attached to the user's mouth. The breathing information extraction filter 300 includes band path filter, high path filter, low path filter, and/or a filter that calculates root-mean-square (RMS) envelope of input signal. The breathing information evaluation filter 302 may include peak detection filters based on traditional window-threshold technique, wavelet transform, Hilbert transform, artificial neural networks, techniques using templates, morphology filtering, nonlinear filtering, Kalman filtering, Gabor filtering, Gaussian second derivative filtering, linear prediction analysis, higher-order statistics, K-Means clustering, fuzzy C-Means clustering, Empirical Mode Decomposition, hidden Markov models, Savitzky-Golay filtering, and smoothed nonlinear energy operator. The breathing monitoring filter 304 includes the filter that finds the breathing rate out of normal range. A breathing information transmission circuit 306 can transmit the respiration information to a remote data storage device in a cloud computing environment.

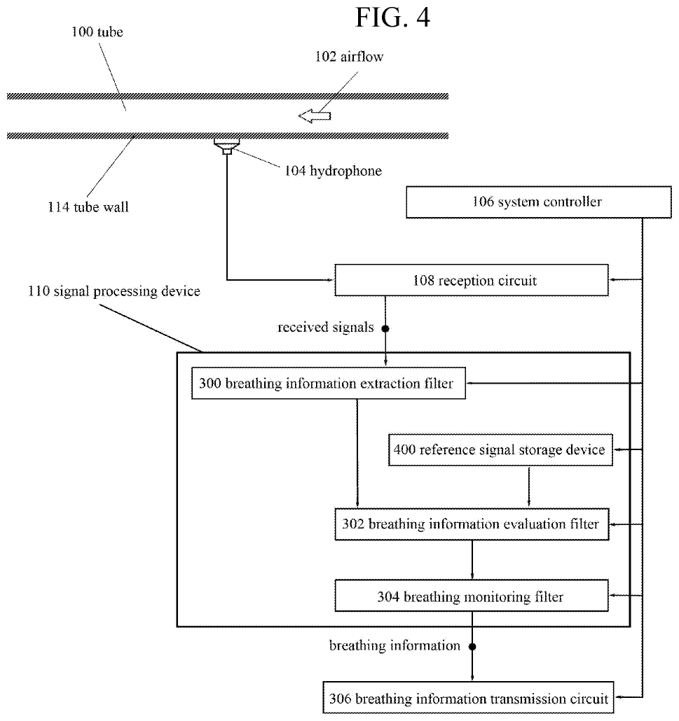

In an embodiment, a reference signal storage device 400 can be used. FIG. 4 shows a schematic diagram of a breathing monitoring apparatus that estimates breathing pattern, respiratory rate, presence of breathing, and/or the air volume flowing in a tube per unit of time, comprising: one or plural hydrophones 104 with one or plural reception circuits 108 that convert a plurality of sounds transmitted through a tube wall 114 and/or inside a tube 100 to a plurality of received signals; a breathing information extraction filter 300 that extracts breathing information from a plurality of received signals acquired by one or plural hydrophones 104 with one or plural reception circuits 108; a breathing information evaluation filter 302 that evaluates breathing pattern, respiratory rate, presence of breathing, and/or the airflow in a tube using the breathing information extracted by a breathing information extraction filter 300 and reference signals stored by a reference signal storage device 400, a breathing monitoring filter 304 that monitors breathing pattern, respiratory rate, presence of breathing, and/or the airflow in a tube; and a breathing information transmission circuit 306 that transmits the information related to respiration including breathing pattern, respiratory rate, and/or presence of breathing. The signal processing device 110 includes the breathing information extraction filter 300, the breathing information evaluation filter 302, the breathing monitoring filter 304, and the reference signal storage device 400. Reference signals include the waveforms of breathing sounds. A computer, that includes central processing unit (CPU) and a memory such as read-only memory (ROM) and random access memory (RAM), may include both the system controller 106 and the signal processing device 110, where the signal processing device 110 includes the reference signal storage device 400.

The reference signal storage device 400 and/or the breathing information evaluation filter 302 may update reference signals and evaluation method after installing the breathing monitoring apparatus in order to adjust the setting of a breathing monitoring apparatus suitable for the installation environment.

The reference signal storage device 400 may store plural reference signals for different respiratory volumes, and the breathing information evaluation filter 302 evaluates respiratory volume using breathing information extracted by the breathing information extraction filter 300 and reference signals stored by the reference signal storage device 400. The breathing information evaluation filter 302 may estimate the risk of exacerbation in diseases of a user, because the increase of respiratory volume indicates the possibility of exacerbation in disease of the user. The breathing information evaluation filter 302 may update evaluation method in order to take account of the setting of one or plural hydrophones, tube length, temperature, humidity, and positional relationship between a pumping device and a user.

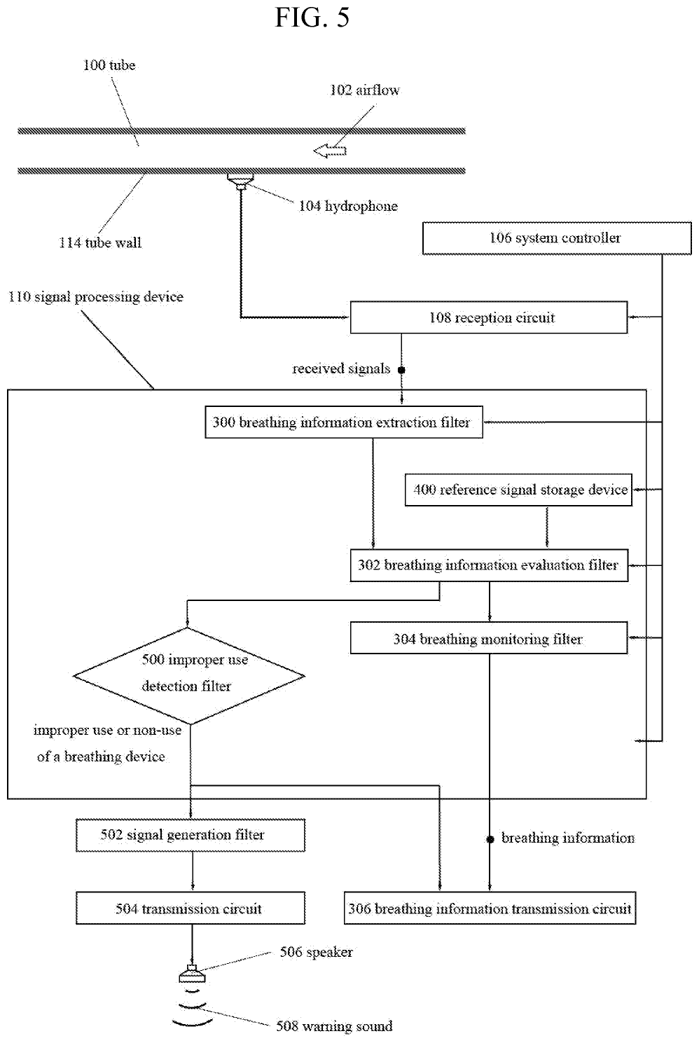

The breathing information transmission circuit 306 may transmit alert information and/or emit warning signal including warning sound, vibration, and light when a breathing information evaluation block detects improper use or non-use of a breathing device. FIG. 5 shows a schematic diagram of a breathing monitoring apparatus that estimates breathing pattern, respiratory rate, presence of breathing, and/or the air volume flowing in a tube per unit of time, comprising: one or plural hydrophones 104 with one or plural reception circuits 108 that convert a plurality of sounds transmitted through a tube wall 114 and/or inside a tube 100 to a plurality of received signals; a breathing information extraction filter 300 that extracts breathing information from a plurality of received signals acquired by one or plural hydrophones 104 with one or plural reception circuits 108; a reference signal storage block 400 that stores one or plural signals related to sounds transmitted through a tube wall 114 and/or inside a tube 100; a breathing information evaluation filter 302 that evaluates breathing pattern, respiratory rate, presence of breathing, and/or the airflow in a tube using breathing information extracted by a breathing information extraction filter 300 and reference signals stored by a reference signal storage device 400; a breathing monitoring filter 302 that monitors breathing pattern, respiratory rate, and/or presence of breathing using the airflow data evaluated from breathing information; and a breathing information transmission circuit 306 that transmits the information related to respiration including breathing pattern, respiratory rate, and/or presence of breathing; an improper use detection filter 500 that detects improper use or non-use of a breathing monitoring apparatus; a signal generation filter 502 that generates a signal for warning sound; a speaker 506 with a transmission circuit 504 that emits waring signal including warning sound 508, vibration and light when the circuitry detects improper use or non-use of a breathing device. When the warning is transmitted, the user, his/her attending physician and/or caregiver can adjust the device and/or provide treatment to the user.

A flow monitoring apparatus according to an embodiment of the present invention includes an oxygen therapy monitoring apparatus that estimates breathing pattern, respiratory rate, presence of breathing, and/or the air volume flowing in a tube per unit of time, comprising: one or plural hydrophones 104 with one or plural reception circuits 108 that convert a plurality of sounds transmitted through a tube wall 114 and/or inside a tube 100 to a plurality of received signals; a breathing information extraction filter 300 that extracts breathing information from a plurality of received signals acquired by one or plural hydrophones 104 with one or plural reception circuits 108; a breathing information evaluation filter 302 that evaluates breathing pattern, respiratory rate, presence of breathing, and/or the airflow in a tube using the breathing information extracted by a breathing information extraction filter 300 and reference signals stored by a reference signal storage device 400, a breathing monitoring filter 304 that monitors breathing pattern, respiratory rate, presence of breathing, and/or the airflow in a tube.

An oxygen therapy monitoring apparatus according to an embodiment of the present invention can be used to estimate breathing pattern, respiratory rate, presence of breathing, the air volume flowing in a tube per unit of time and/or air leakage, comprising: one or plural hydrophones 104 with one or plural reception circuits 108 that convert a plurality of sounds transmitted through a tube wall and/or inside a tube to a plurality of received signals; a breathing information extraction filter 300 that extracts breathing information from a plurality of received signals acquired by one or plural hydrophones 104 with one or plural reception circuits 108; a reference signal storage device 400 that stores plural reference signals for different respiratory volumes and/or for air leakage conditions; a breathing information evaluation filter 302 that evaluates breathing pattern, respiratory rate, presence of breathing, the airflow in a tube and/or air leakage using breathing information extracted by a breathing information extraction block 300 and reference signals stored by a reference signal storage device 400; a breathing monitoring filter 304 that monitors breathing pattern, respiratory rate, presence of breathing, the airflow in a tube and/or air leakage; and a breathing information transmission circuit 306 that transmits the information related to respiration including breathing pattern, respiratory rate, presence of breathing, the airflow in a tube and/or air leakage.

An oxygen therapy monitoring apparatus can be used to estimate one or plural air leakage locations, wherein a breathing information evaluation filter 302 estimates one or plural air leakage locations.

A flow monitoring apparatus according to an embodiment of the present invention includes a non-invasive ventilation therapy monitoring apparatus that estimates breathing pattern, respiratory rate, presence of breathing, and/or the air volume flowing in a tube per unit of time, comprising: one or plural hydrophones 104 with one or plural reception circuits 108 that convert a plurality of sounds transmitted through a tube wall 114 and/or inside a tube 100 to a plurality of received signals; a breathing information extraction filter 300 that extracts breathing information from a plurality of received signals acquired by one or plural hydrophones 104 with one or plural reception circuits 108; a breathing information evaluation filter 302 that evaluates breathing pattern, respiratory rate, presence of breathing, and/or the airflow in a tube using the breathing information extracted by a breathing information extraction filter 300; a breathing monitoring filter 304 that monitors breathing pattern, respiratory rate, presence of breathing, and/or the airflow in a tube; and a breathing information transmission circuit 306 that transmits the information related to respiration including breathing pattern, respiratory rate, presence of breathing, and/or airflow in a tube.

In an embodiment, a non-invasive ventilation therapy monitoring apparatus can be used to estimate breathing pattern, respiratory rate, presence of breathing, the air volume flowing in a tube per unit of time and/or air leakage, comprising: one or plural hydrophones 104 with one or plural reception circuits 108 that convert a plurality of sounds transmitted through a tube wall 114 and/or inside a tube 100 to a plurality of received signals; a breathing information extraction filter 300 that extracts breathing information from a plurality of received signals acquired by one or plural hydrophones 104 with one or plural reception circuits 108; a reference signal storage device 400 that stores plural reference signals for different respiratory volumes and/or for air leakage conditions; a breathing information evaluation filter 302 that evaluates breathing pattern, respiratory rate, presence of breathing, the airflow in a tube and/or air leakage using breathing information extracted by a breathing information extraction filter 300 and reference signals stored by a reference signal storage device 400; a breathing monitoring filter 304 that monitors breathing pattern, respiratory rate, presence of breathing, the airflow in a tube and/or air leakage; and a breathing information transmission circuit 306 that transmits the information related to respiration including breathing pattern, respiratory rate, presence of breathing, the airflow in a tube and/or air leakage.

A non-invasive ventilation therapy monitoring apparatus can be used to evaluate one or plural air leakage locations, wherein a breathing information evaluation filter 302 estimates one or plural air leakage locations.

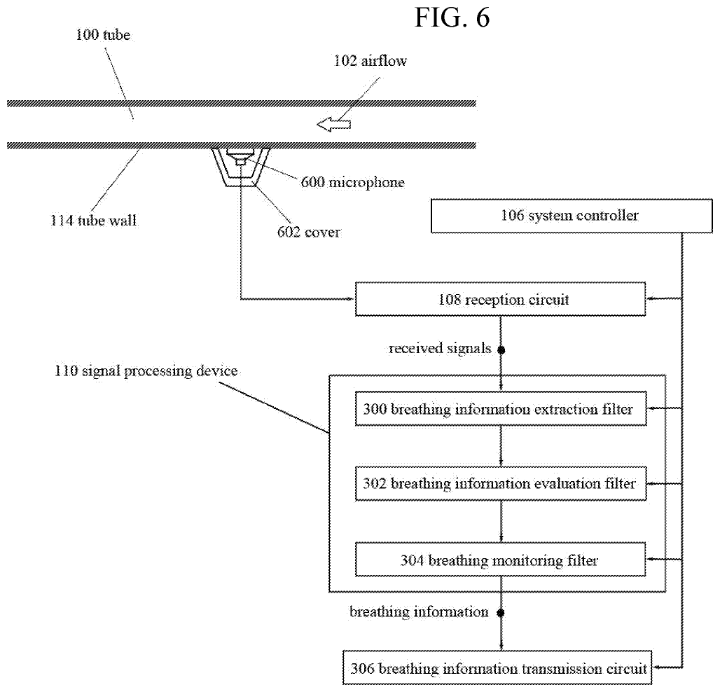

A flow monitoring apparatus according to an embodiment of the present invention can employ one or plural microphones as a substitute of one or plural hydrophones. One or plural microphones with one or plural covers can be employed as a substitute of one or plural hydrophones. FIG. 6 shows a schematic diagram of a breathing monitoring apparatus that employ a microphone 600 with a cover 602 as a substitute of a hydrophone. Flow monitoring apparatus of the present invention can employ one or plural hydrophones with one or plural covers as a substitute of one or plural hydrophones.

### First Exemplary Embodiment

FIG. 4 shows a schematic diagram of a breathing monitoring apparatus that estimates breathing pattern, respiratory rate, presence of breathing, and/or the air volume flowing in a tube per unit of time, comprising: one or plural hydrophones 104 with one or plural reception circuits 108 that convert a plurality of sounds transmitted through a tube wall 114 and/or inside a tube 100 to a plurality of received signals; a breathing information extraction filter 300 that extracts breathing information from a plurality of received signals acquired by one or plural hydrophones 104 with one or plural reception circuits 108; a breathing information evaluation filter 302 that evaluates breathing pattern, respiratory rate, presence of breathing, and/or the airflow in a tube using the breathing information extracted by a breathing information extraction filter 300 and reference signals stored by a reference signal storage device 400, a breathing monitoring filter 304 that monitors breathing pattern, respiratory rate, presence of breathing, and/or the airflow in a tube; and a breathing information transmission circuit 306 that transmits the information related to respiration including breathing pattern, respiratory rate, and/or presence of breathing. Reference signals include the waveforms of breathing sounds. The breathing information extraction filter 300 comprises a band path filter followed by a root-mean-square (RMS) envelope filter. The breathing information evaluation filter 302 consists of a peak detection filter based on traditional window-threshold technique. The breathing monitoring filter 304 includes the filter that finds the breathing rate out of normal range. A breathing information transmission circuit 306 can transmit the respiration information to a remote data storage device in a cloud computing environment.

### Second Exemplary Embodiment

FIG. 5 shows a schematic diagram of a breathing monitoring apparatus that estimates breathing pattern, respiratory rate, presence of breathing, and/or the air volume flowing in a tube per unit of time, comprising: one or plural hydrophones 104 with one or plural reception circuits 108 that convert a plurality of sounds transmitted through a tube wall 114 and/or inside a tube 100 to a plurality of received signals; a breathing information extraction filter 300 that extracts breathing information from a plurality of received signals acquired by one or plural hydrophones 104 with one or plural reception circuits 108; a reference signal storage block 400 that stores one or plural signals related to sounds transmitted through a tube wall 114 and/or inside a tube 100; a breathing information evaluation filter 302 that evaluates breathing pattern, respiratory rate, presence of breathing, and/or the airflow in a tube using breathing information extracted by a breathing information extraction filter 300 and reference signals stored by a reference signal storage device 400; a breathing monitoring filter 302 that monitors breathing pattern, respiratory rate, and/or presence of breathing using the airflow data evaluated from breathing information; and a breathing information transmission circuit 306 that transmits the information related to respiration including breathing pattern, respiratory rate, and/or presence of breathing; an improper use detection filter 500 that detects improper use or non-use of a breathing monitoring apparatus; a signal generation filter 502 that generates a signal for warning sound; a speaker 506 with a transmission circuit 504 that emits waring signal including warning sound 508, vibration and light when the circuitry detects improper use or non-use of a breathing device. When the warning is transmitted, the user, his/her attending physician and/or caregiver can adjust the device and/or provide treatment to the user.

### Third Exemplary Embodiment

A non-invasive ventilation therapy monitoring apparatus can be used to estimate breathing pattern, respiratory rate, presence of breathing, the air volume flowing in a tube per unit of time and/or air leakage, comprising: one or plural hydrophones 104 with one or plural reception circuits 108 that convert a plurality of sounds transmitted through a tube wall 114 and/or inside a tube 100 to a plurality of received signals; a breathing information extraction filter 300 that extracts breathing information from a plurality of received signals acquired by one or plural hydrophones 104 with one or plural reception circuits 108; a reference signal storage device 400 that stores plural reference signals for different respiratory volumes and/or for air leakage conditions; a breathing information evaluation filter 302 that evaluates breathing pattern, respiratory rate, presence of breathing, the airflow in a tube and/or air leakage using breathing information extracted by a breathing information extraction filter 300 and reference signals stored by a reference signal storage device 400; a breathing monitoring filter 304 that monitors breathing pattern, respiratory rate, presence of breathing, the airflow in a tube and/or air leakage; and a breathing information transmission circuit 306 that transmits the information related to respiration including breathing pattern, respiratory rate, presence of breathing, the airflow in a tube and/or air leakage. A controller comprising circuitry may include reception circuit 108 and a breathing information transmission circuit 306.

The present invention has the following aspects.

1. A flow monitoring apparatus, comprising: one or plural hydrophones that receive a plurality of sounds transmitted through a tube wall and/or inside a tube; and a controller comprising circuitry configured to convert the plurality of sounds received by one or plural hydrophones to a plurality of received signals, extract flow information from the plurality of received signals, evaluate flow rate in a tube, and transmit the information of volume flowing in a tube per unit of time.

2. An airflow monitoring apparatus, comprising: one or plural hydrophones that receive a plurality of sounds transmitted through a tube wall and/or inside a tube; and a controller comprising circuitry configured to convert the plurality of sounds received by one or plural hydrophones to a plurality of received signals, extract airflow information from the plurality of received signals, evaluate airflow rate in a tube, and transmit the information of air volume flowing in a tube per unit of time.

3. A breathing monitoring apparatus, comprising: one or plural hydrophones that receive a plurality of sounds transmitted through a tube wall and/or inside a tube; and a controller comprising circuitry configured to convert the plurality of sounds received by one or plural hydrophones to a plurality of received signals, extract breathing information from the plurality of received signals, evaluate breathing pattern, respiratory rate, presence of breathing, and/or the airflow in a tube using the breathing information, monitor breathing pattern, respiratory rate, presence of breathing, and/or the airflow in a tube, and transmit the information related to respiration including breathing pattern, respiratory rate, and/or presence of breathing.

4. The breathing monitoring apparatus according to 3, wherein the circuitry further configured to transmit the respiration information to a remote data storage device in a cloud computing environment.

5. A breathing monitoring apparatus, comprising: one or plural hydrophones that receive a plurality of sounds transmitted through a tube wall and/or inside a tube; and a controller comprising circuitry configured to convert the plurality of sounds received by one or plural hydrophones to a plurality of received signals, extract breathing information from the plurality of received signals, store one or plural reference signals related to sounds transmitted through a tube wall and/or inside a tube, evaluate breathing pattern, respiratory rate, presence of breathing, and/or the airflow in a tube using the breathing information and the reference signals, monitor breathing pattern, respiratory rate, and/or presence of breathing using the airflow data evaluated from the breathing information, and transmits the information related to respiration including breathing pattern, respiratory rate, and/or presence of breathing.

6. The breathing monitoring apparatus according to 5, wherein the circuitry further configured to update the reference signals after installing the breathing monitoring apparatus.

7. The breathing monitoring apparatus according to 5, wherein the circuitry further configured to update the evaluation method after installing the breathing monitoring apparatus.

8. The breathing monitoring apparatus according to 5, wherein the circuitry configured to store plural reference signals for different respiratory volumes, and evaluate respiratory volume using the breathing information and the reference signals.

9. The breathing monitoring apparatus according to 8, wherein the circuitry further configured to estimate the risk of exacerbation in diseases of a user.

10. The breathing monitoring apparatus according to 7, wherein the circuitry further configured to update evaluation method in order to take account of the setting of one or plural hydrophones, tube length, temperature, humidity, and positional relationship between a pumping device and a user.

11. The breathing monitoring apparatus according to 3, wherein the circuitry further configured to transmit alert information and/or emit warning signal including warning sound, vibration and light when the circuitry detects improper use or non-use of a breathing device.

12. An oxygen therapy monitoring apparatus, comprising: one or plural hydrophones that receive a plurality of sounds transmitted through a tube wall and/or inside a tube; and a controller comprising circuitry configured to convert the plurality of sounds received by one or plural hydrophones to a plurality of received signals, extract breathing information from a plurality of the received signals, evaluate breathing pattern, respiratory rate, presence of breathing, and/or the airflow in a tube using the breathing information, monitor breathing pattern, respiratory rate, presence of breathing, and/or the airflow in a tube, and transmit the information related to respiration including breathing pattern, respiratory rate, presence of breathing, and/or airflow in a tube.

13. An oxygen therapy monitoring apparatus, comprising: one or plural hydrophones that receive a plurality of sounds transmitted through a tube wall and/or inside a tube; and a controller comprising circuitry configured to convert the plurality of sounds received by one or plural hydrophones to a plurality of received signals, extract breathing information from a plurality of the received signals, store plural reference signals for different respiratory volumes and/or for air leakage conditions, evaluate breathing pattern, respiratory rate, presence of breathing, the airflow in a tube and/or air leakage using the breathing information and the reference signals, monitor breathing pattern, respiratory rate, presence of breathing, the airflow in a tube and/or air leakage, and transmit the information related to respiration including breathing pattern, respiratory rate, presence of breathing, the airflow in a tube and/or air leakage.

14. The oxygen therapy monitoring apparatus according to 13, wherein the circuitry further configured to estimate one or plural air leakage locations.

15. A non-invasive ventilation therapy monitoring apparatus, comprising: one or plural hydrophones that receive a plurality of sounds transmitted through a tube wall and/or inside a tube; and a controller comprising circuitry configured to convert the plurality of sounds received by one or plural hydrophones to a plurality of received signals, extract breathing information from a plurality of the received signals, evaluate breathing pattern, respiratory rate, presence of breathing, and/or the airflow in a tube using the breathing information, monitor breathing pattern, respiratory rate, presence of breathing, and/or the airflow in a tube, and transmit the information related to respiration including breathing pattern, respiratory rate, presence of breathing, and/or airflow in a tube.

16. A non-invasive ventilation therapy monitoring apparatus, comprising: one or plural hydrophones that receive a plurality of sounds transmitted through a tube wall and/or inside a tube; and a controller comprising circuitry configured to convert the plurality of sounds received by one or plural hydrophones to a plurality of received signals, extract breathing information from a plurality of the received signals, store plural reference signals for different respiratory volumes and/or for air leakage conditions, evaluate breathing pattern, respiratory rate, presence of breathing, the airflow in a tube and/or air leakage using the breathing information and the reference signals, monitor breathing pattern, respiratory rate, presence of breathing, the airflow in a tube and/or air leakage, and transmit the information related to respiration including breathing pattern, respiratory rate, presence of breathing, the airflow in a tube and/or air leakage.

17. The non-invasive ventilation therapy monitoring apparatus according to 16, wherein the circuitry further configured to estimate one or plural air leakage locations.

18. A breathing monitoring apparatus, including: at least one microphone that receives a plurality of sounds transmitted through a tube wall and/or inside a tube; and a controller comprising circuitry configured to convert the plurality of sounds received by one or plural microphones to a plurality of received signals, extract breathing information from the plurality of received signals, evaluate breathing pattern, respiratory rate, presence of breathing, and/or the airflow in a tube using the breathing information, monitor breathing pattern, respiratory rate, presence of breathing, and/or the airflow in a tube, and transmit the information related to respiration including breathing pattern, respiratory rate, and/or presence of breathing.

19. The breathing monitoring apparatus according to 18, wherein the breathing monitoring apparatus includes one or plural microphones with one or plural covers that receives a plurality of sounds transmitted through a tube wall and/or inside a tube.

20. The apparatus according to 1, 2, 3, 5, 10, 12, 13, 15 and 16, wherein one or plural hydrophones with one or plural covers are employed as a substitute of the one or plural hydrophones.

Obviously, numerous modifications and variations of the present invention are possible in light of the above teachings. It is therefore to be understood that within the scope of the appended claims, the invention may be practiced otherwise than as specifically described herein.

### REFERENCE SIGNS LIST

100 tube
102 airflow
104 hydrophone
106 system controller
108 reception circuit
110 signal processing device
112 airflow information transmission circuit
114 tube wall
200 airflow information extraction filter
202 airflow rate evaluation filter
300 breathing information extraction filter
302 breathing information evaluation filter
304 breathing monitoring filter
306 breathing information transmission circuit
400 reference signal storage device
500 improper use detection filter
502 signal generation filter
504 transmission circuit
506 speaker
508 warning sound
600 microphone
602 cover

What is claimed is:

1. A flow monitoring apparatus, comprising:
at least one hydrophone that receives a plurality of sounds transmitted through a tube wall and/or inside a tube; and
a controller comprising circuitry configured to convert the sounds received by the at least one hydrophone to a plurality of received signals, extract flow information from the received signals, evaluate flow rate in a tube, and transmit the information of volume flowing in a tube per unit of time.

2. The flow monitoring apparatus according to claim 1, further comprising:
at least one cover that covers the at least one hydrophone.

3. An airflow monitoring apparatus, comprising:
at least one hydrophone that receives a plurality of sounds transmitted through a tube wall and/or inside a tube; and
a controller comprising circuitry configured to convert the sounds received by the at least one hydrophone to a plurality of received signals, extract airflow information from the received signals, evaluate airflow rate in a tube, and transmit the information of air volume flowing in a tube per unit of time.

4. A breathing monitoring apparatus, comprising:
at least one hydrophone that receives a plurality of sounds transmitted through a tube wall and/or inside a tube; and
a controller comprising circuitry configured to convert the sounds received by the at least one hydrophone to a plurality of received signals, extract breathing information from the received signals, evaluate breathing pattern, respiratory rate, presence of breathing, and/or the airflow in a tube using the breathing information, monitor breathing pattern, respiratory rate, presence of breathing, and/or the airflow in a tube, and transmit the information related to respiration including breathing pattern, respiratory rate, and/or presence of breathing.

5. The breathing monitoring apparatus according to claim 4, wherein the circuitry is further configured to transmit the respiration information to a remote data storage device in a cloud computing environment.

6. The breathing monitoring apparatus according to claim 4, wherein the circuitry is further configured to transmit alert information and/or emit warning signal including warning sound, vibration and light when the circuitry detects improper use or non-use of a breathing device.

7. A breathing monitoring apparatus, comprising:
at least one hydrophone that receives a plurality of sounds transmitted through a tube wall and/or inside a tube; and
a controller comprising circuitry configured to convert the sounds received by the at least one hydrophone to a plurality of received signals, extract breathing information from the received signals, store one or plural reference signals related to sounds transmitted through a tube wall and/or inside a tube, evaluate breathing pattern, respiratory rate, presence of breathing, and/or the airflow in a tube using the breathing information and the reference signals, monitor breathing pattern, respiratory rate, and/or presence of breathing using the airflow data evaluated from the breathing information, and transmits the information related to respiration including breathing pattern, respiratory rate, and/or presence of breathing.

8. The breathing monitoring apparatus according to claim 7, wherein the circuitry is further configured to update the reference signals after installing the breathing monitoring apparatus.

9. The breathing monitoring apparatus according to claim 7, wherein the circuitry is further configured to update an evaluation method after installing the breathing monitoring apparatus.

10. The breathing monitoring apparatus according to claim 7, wherein the circuitry is configured to store plural reference signals for different respiratory volumes, and evaluate respiratory volume using the breathing information and the reference signals.

11. The breathing monitoring apparatus according to claim 10, wherein the circuitry is further configured to estimate risk of exacerbation in diseases of a user.

12. The breathing monitoring apparatus according to claim 9, wherein the circuitry is further configured to update an evaluation method to take account of setting of the at least one hydrophone, a tube length, a temperature, humidity, and a positional relationship between a pumping device and a user.

13. An oxygen therapy monitoring apparatus, comprising:
at least one hydrophone that receives a plurality of sounds transmitted through a tube wall and/or inside a tube; and
a controller comprising circuitry configured to convert the sounds received by the at least one hydrophone to a plurality of received signals, extract breathing information from a plurality of the received signals, evaluate breathing pattern, respiratory rate, presence of breathing, and/or the airflow in a tube using the breathing information, monitor breathing pattern, respiratory rate, presence of breathing, and/or the airflow in a tube, and transmit the information related to respiration including breathing pattern, respiratory rate, presence of breathing, and/or airflow in a tube.

14. An oxygen therapy monitoring apparatus, comprising:
at least one hydrophone that receives a plurality of sounds transmitted through a tube wall and/or inside a tube; and
a controller comprising circuitry configured to convert the sounds received by the at least one hydrophone to a plurality of received signals, extract breathing information from the received signals, store plural reference signals for different respiratory volumes and/or for air leakage conditions, evaluate breathing pattern, respiratory rate, presence of breathing, the airflow in a tube and/or air leakage using the breathing information and the reference signals, monitor breathing pattern, respiratory rate, presence of breathing, the airflow in a tube and/or air leakage, and transmit the information related to respiration including breathing pattern, respiratory rate, presence of breathing, the airflow in a tube and/or air leakage.

15. The oxygen therapy monitoring apparatus according to claim 14, wherein the circuitry is further configured to estimate one or plural air leakage locations.

16. A non-invasive ventilation therapy monitoring apparatus, comprising:
at least one hydrophone that receives a plurality of sounds transmitted through a tube wall and/or inside a tube; and
a controller comprising circuitry configured to convert the sounds received by the at least one hydrophone to a plurality of received signals, extract breathing information from a plurality of the received signals, evaluate breathing pattern, respiratory rate, presence of breathing, and/or the airflow in a tube using the breathing information, monitor breathing pattern, respiratory rate, presence of breathing, and/or the airflow in a tube, and transmit the information related to respiration including breathing pattern, respiratory rate, presence of breathing, and/or airflow in a tube.

17. A non-invasive ventilation therapy monitoring apparatus, comprising:
at least one hydrophone that receives a plurality of sounds transmitted through a tube wall and/or inside a tube; and
a controller comprising circuitry configured to convert the sounds received by the at least one hydrophone to a plurality of received signals, extract breathing information from the received signals, store plural reference signals for different respiratory volumes and/or for air leakage conditions, evaluate breathing pattern, respiratory rate, presence of breathing, the airflow in a tube and/or air leakage using the breathing information and the reference signals, monitor breathing pattern, respiratory rate, presence of breathing, the airflow in a tube and/or air leakage, and transmit the information related to respiration including breathing pattern, respiratory rate, presence of breathing, the airflow in a tube and/or air leakage.

18. The non-invasive ventilation therapy monitoring apparatus according to claim 17, wherein the circuitry is further configured to estimate at least one air leakage location.

19. A breathing monitoring apparatus, comprising:
at least one microphone that receives a plurality of sounds transmitted through a tube wall and/or inside a tube; and
a controller comprising circuitry configured to convert the sounds received by the at least one microphone to a plurality of received signals, extract breathing information from the received signals, evaluate breathing pattern, respiratory rate, presence of breathing, and/or the airflow in a tube using the breathing information, monitor breathing pattern, respiratory rate, presence of breathing, and/or the airflow in a tube, and transmit the information related to respiration including breathing pattern, respiratory rate, and/or presence of breathing.

20. The breathing monitoring apparatus according to claim 19, further comprising:
at least one cover that covers the at least one microphone.
